(19) United States
(12) Patent Application Publication
(10) Pub. No.: US 2012/0251479 A1
(43) Pub. Date: Oct. 4, 2012

(54) Reduced-Odor Polyol Composition And Method Of Producing Same

(76) Inventors: Chacko Thankachan, West Bloomfield, MI (US); Jay G. Otten, Flat Rock, MI (US); Ernestine Satchell, Ecorse, MI (US)

(21) Appl. No.: 13/499,838

(22) PCT Filed: Sep. 28, 2010
(86) PCT No.: PCT/US10/50524

§ 371 Date: Jun. 19, 2012

Related U.S. Application Data
(60) Provisional application No. 61/248,033, filed on Oct. 2, 2009.

(51) Int. Cl. A61K 31/77; A61P 1/00

(52) U.S. Cl. 424/78.01

(57) ABSTRACT

A reduced-odor polyol composition for gastrointestinal lavage of a mammal comprises a polyol and at least one acid salt formed from a reaction between a basic catalyst and citric acid. The reduced-odor polyol composition is produced by providing at least one alkylene oxide; providing at least one initiator; reacting the at least one alkylene oxide with the at least one initiator in the presence of a basic catalyst to produce a polyol; and neutralizing the basic catalyst with citric acid, thereby forming at least one acid salt and producing the reduced-odor polyol composition.

### FIELD OF THE INVENTION

The invention generally relates to a reduced-odor polyol composition and, more particularly, to a method of producing a reduced-odor polyol composition for gastrointestinal lavage for a mammal.

### DESCRIPTION OF THE RELATED ART

Polyols and methods of producing polyols are well known in the art. Polyols are generally defined as carbon-based compounds having two or more hydroxy functional groups, and are utilized in a variety of applications. For example, a common application in which polyols are utilized is the production of polyurethanes/polyisocyanurates via a reaction of the polyol with an isocyanate.

One type of polyol is polyether polyol, which has a backbone of repeating ether units, i.e., R-O-R units, where R can be the same or different, and two or more hydroxy functional groups. Polyether polyols are produced by reacting alkylene oxides, such as ethylene oxide (EO), propylene oxide (PO), and/or butylene oxide (BO) with an initiator in the presence of a catalyst. For example, when the initiator utilized is ethylene glycol and ethylene oxide is utilized to form the backbone of repeating ether units, the resulting polyether polyol is a polyethylene glycol (PEG). Common catalysts include metal cyanide catalysts and strong basic catalysts, such as potassium hydroxide (KOH), sodium hydroxide (NaOH), and/or cesium hydroxide (CsOH). Because such catalysts can have adverse affects on subsequent reactions and/or applications utilizing the polyether polyols, it is advantageous to neutralize the catalysts and/or remove the catalysts from the polyether polyols. For example, when the catalyst is the strong basic catalyst, the strong basic catalyst is removed by absorbent treatment and filtration or is neutralized with an acid, which results in the production of an acid salt. When the polyether polyol is a liquid, the acid salt may precipitate, and can be removed from the polyether polyol by several methods, such as by filtration. Neutralizing the strong basic catalyst with the acid neutralizes the pH of a composition including the polyether polyol, which makes the polyether polyol suitable for diverse applications.

Because polyether polyols are non-ionic and non-toxic, another application in which polyether polyols may be utilized is pharmaceutical excipients and active pharmaceutical ingredients (API). For example, polyether polyols may be utilized as a laxative, or a gastrointestinal lavage. A lavage is a process by which an organ is “washed out” or “flushed” with a liquid. For example, polyethylene glycol, which is one type of polyether polyol, may be dissolved in water and consumed orally by a patient in preparation for a colonoscopy. The polyethylene glycol operates as a laxative and cleanses a gastrointestinal tract of the patient for the colonoscopy or other gastrointestinal procedure.

Polyethylene glycol is produced by reacting ethylene glycol with ethylene oxide in the presence of a catalyst. When the polyethylene glycol is produced for the purposes of the lavage, the catalyst is the strong basic catalyst because the metal cyanide catalyst could have potentially adverse health impacts when consumed orally by the patient. In addition, the strong basic catalyst is neutralized with the acid so that the polyethylene glycol does not have a basic pH, which is also undesirable for oral consumption.

Currently, the strong basic catalyst is neutralized with a combination of acetic acid and phosphoric acid, which produces acid salts. These acid salts are not removed from the polyethylene glycol, and disassociate into respective ions when the polyethylene glycol is dissolved in water prior to being orally consumed by the patient. In addition, because one of the acids utilized to neutralize the basic catalyst is acetic acid, residual odors often exist in the polyether polyol that are undesirable when the polyether polyol is to be orally consumed by the patient.

Further, polyether polyols, including polyethylene glycols, are susceptible to oxidative degradation, which results in an undesirable odor. The undesirable odor makes it difficult for patients to orally consume the polyethylene glycol. To mitigate these undesirable effects, an antioxidant, such butylated hydroxytoluene (BHT), is typically incorporated into the polyether polyols to prevent such oxidative degradation of the polyethylene glycol. However, incorporation of antioxidants is not permitted for many applications of polyether polyols. When permitted, the antioxidant must be precisely added to the polyethylene glycol for adherence to quality standards. For example, in liquid polyether polyols, if the polyether polyols are not slightly acidic when the antioxidant is added to the polyether polyols, a pink hue develops in the polyether polyols, which is attributable to the formation of a phenoxide salt. When the polyether polyols develop a pink hue, the polyether polyols are typically not regarded as being of high enough quality for oral consumption. In solid polyether polyols, a yellow hue may develop on the surfaces of the polyether polyols, which is attributable to the formation of quinone compounds. When the polyether polyols develops a yellow hue, the polyether polyols are similarly rejected for quality concerns. As such, it is difficult to produce polyether polyols for oral consumption that meet quality standards when antioxidants are required. Further, addition of the antioxidant introduces additional costs and processing steps into the method of producing polyether polyols.

### SUMMARY OF THE INVENTION

The present invention provides a reduced-odor polyol composition for gastrointestinal lavage of a mammal. The reduced-odor polyol composition comprises a polyol and at least one acid salt formed from a reaction between a basic catalyst and citric acid. The present invention also provides a method of producing the reduced-odor polyol composition. The method of producing the reduced-odor polyol composition comprises the steps of providing at least one alkylene oxide; providing at least one initiator; reacting the at least one alkylene oxide with the at least one initiator in the presence of a basic catalyst to produce a polyol; and neutralizing the basic catalyst with citric acid, thereby forming at least one acid salt and producing the reduced-odor polyol composition. The present invention further provides a method of treating a mammal with a reduced-odor polyol composition. The method of treating the mammal with the reduced-odor polyol composition comprises the steps of providing the reduced-odor polyol composition and administering the reduced-odor polyol composition to treat the mammal.

The reduced-odor polyol composition has an improved dissolution rate in water when compared with other conventional polyol compositions which have been neutralized by acids other than citric acid. In addition, the reduced-odor polyol composition of the present invention has excellent resistance to oxidative degradation, even when the reduced-odor polyol composition is free from antioxidants other than the acid salt, which results in a minimal to non-existent odor from such oxidative degradation. As such, the reduced-odor polyol composition is more suitable for oral consumption by patients because the reduced-odor polyol composition does not have an undesirable odor which is commonly associated with conventional polyol compositions.

### DETAILED DESCRIPTION OF THE INVENTION

The present invention provides a reduced-odor polyol composition and a method of producing the reduced-odor polyol composition, each of which is described in greater detail below. The reduced-odor polyol composition of the present invention is particularly suitable for gastrointestinal lavage of a mammal, such as a human. However, it is to be appreciated that the reduced-odor polyol composition of the present invention is not limited gastrointestinal lavage; for example, the reduced-odor polyol composition could be utilized in a reaction with an isocyanate to produce a polyurethane foam or elastomer.

It is known throughout that art that polyols, including polyether polyols, are susceptible to oxidative degradation. Such oxidative degradation results in, among other undesirable effects, unpleasant odors. The terminology “reduced-odor”, in reference to the reduced-odor polyol composition of the present invention, is relative to conventional polyols. Stated differently, the reduced-odor polyol composition of the present invention has excellent resistance to oxidative degradation and, as such, has a reduced-odor when compared with conventional polyols, which are susceptible to oxidative degradation.

The reduced-odor polyol composition comprises a polyol. The polyol of the reduced-odor polyol composition may be any polyol having two or more hydroxy functional groups per molecule. Further, the polyol can be selected from, but is not limited to, the group of aliphatic polyols, cycloaliphatic polyols, aromatic polyols, heterocyclic polyols, and combinations thereof. The polyol of the reduced-odor polyol composition may be a polyether polyol, i.e., a polyol comprising R-O-R units where R can be the same or different, a polyester polyol, i.e., a polyol comprising R¹-COO-R¹ units where R¹ may be the same or different, or combinations thereof. In certain embodiments, the polyol of the reduced-odor polyol composition is further defined as a polyalkylene glycol, such as a polyethylene glycol, polypropylene glycol, polybutylene glycol, etc. One example of a polyalkylene glycol suitable for the purposes of the present invention is polyethylene glycol (PEG), which is shown in Formula 1 immediately below for illustrative purposes only:

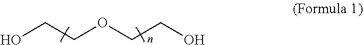

wherein n is an integer greater than 1 depending on a desired molecular weight of the polyethylene glycol.

Although not required, when the polyol of the reduced-odor polyol composition is the polyalkylene glycol, the polyalkylene glycol typically has a nominal average molecular weight of from 3,000 to 3,700, more typically from 3,150 to 3,550, and most typically from 3,300 to 3,400. In other embodiments in which the polyol is other than the polyalkylene glycol, the polyol typically has a nominal average molecular weight within these ranges as well.

The reduced-odor polyol composition further comprises at least one acid salt formed from a reaction between a basic catalyst and citric acid. In certain embodiments, the reduced-odor polyol composition consists essentially of the polyol and the acid salt. It is to be appreciated that the citric acid may be utilized in concert with one or more other types of acids, such as lactic acid. However, although a blend of different types of acids may be utilized, the blend of different types of acids is typically free from acetic acid. Citric acid is shown below in Formula 2 for illustrative purposes only:

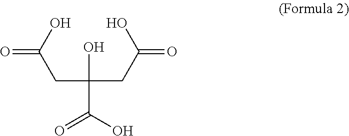

For purposes of clarity, the at least one acid salt is hereinafter referred to as “the acid salt”, which includes embodiments in which the acid salt comprises the blend of different types of acid salts. The structure and type of the acid salt is contingent on the basic catalyst, as well as whether any additional acids are utilized in combination with citric acid, as described in greater detail below. The amount of the acid salt present in the reduced-odor polyol composition is contingent on the amount of the basic catalyst utilized when forming the polyol. The acid salt is typically present in the reduced-odor polyol composition of the present invention in an amount of from greater than zero to 3, more typically from 0.01 to 1.5, percent by weight based on the total weight of the reduced-odor polyol composition.

In one embodiment, the basic catalyst comprises an alkali hydroxide. An alkali hydroxide comprises an alkali metal cation and a hydroxide anion. The alkali hydroxides are lithium hydroxide (LiOH), sodium hydroxide (NaOH), potassium hydroxide (KOH), rubidium hydroxide (RbOH), and cesium hydroxide (CsOH). It is to be appreciated that a single type of alkali hydroxide may be utilized, such as sodium hydroxide, or a combination of different types of alkali hydroxides, such as potassium hydroxide and sodium hydroxide, may be utilized as the basic catalyst for the purposes of the present invention.

Referring back to the acid salt, when the basic catalyst is the alkali hydroxide, the acid salt typically comprises a citrate anion, which is a conjugate base of citric acid, and at least one alkali metal cation. For example, when the alkali hydroxide is sodium hydroxide, the acid salt comprises sodium citrate, which may be present as monosodium citrate, disodium citrate, trisodium citrate, and combinations thereof. Trisodium citrate is shown below in Formula 3 for illustrative purposes only:

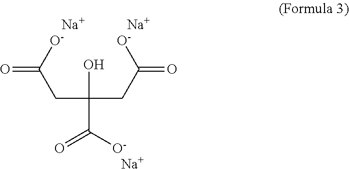

Similarly, when the alkali hydroxide is potassium hydroxide, the acid salt comprises potassium citrate, which may be present as monopotassium citrate, dipotassium citrate, tripotassium citrate, and combinations thereof. Thus, when the basic catalyst comprises the alkali hydroxide, the acid salt is selected from the group of lithium citrate, sodium citrate, potassium citrate, rubidium citrate, cesium citrate, and combinations thereof.

Notably, when the citric acid is utilized in concert with another type of acid, additional types of acid salts are typically present in the reduced-odor polyol composition. For example, when the citric acid is utilized in concert with lactic acid and the basic catalyst is the alkali hydroxide, the acid salt also comprises a lactate anion, which is a conjugate base of lactic acid, and at least one alkali metal cation. For example, when the alkali hydroxide is sodium hydroxide, the acid salt also comprises sodium lactate. Sodium lactate is shown below in Formula 4 for illustrative purposes only:

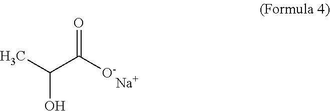

Similarly, when the alkali hydroxide is lithium hydroxide, the acid salt also comprises lithium lactate. Thus, when lactic acid is also utilized, the acid salt may further comprise lithium lactate, sodium lactate, potassium lactate, rubidium lactate, cesium lactate, and combinations thereof.

It is to be appreciated that a blend of different types of basic catalysts may be utilized in forming the acid salt. As such, the acid salts described above are merely examples of the acid salt of the reduced-odor polyol composition, and are in no way representative of an exhaustive list of the acid salts which may be present in the reduced-odor polyol composition.

The reduced-odor polyol composition of the present invention is typically substantially free from antioxidants other than the acid salt and optionally a residual amount of the citric acid and/or the basic catalyst. The acid salt is typically formed in situ in the reduced-odor polyol composition, as described in greater detail below with respect to the method of producing the reduced-odor polyol composition. Therefore, in embodiments in which the acid salt is formed in situ in the reduced-odor polyol composition, a residual amount of the citric acid and/or the basic catalyst may be present in the reduced-odor polyol composition. The residual amount of the citric acid and/or the basic catalyst is typically present in the reduced-odor polyol composition when a molar excess of either the basic catalyst or the citric acid exists relative to the other. Stated differently, if a molar excess of the basic catalyst is utilized relative to the citric acid, a residual amount of the basic catalyst, which is unreacted, i.e., not neutralized, may remain in the reduced-odor polyol composition. Similarly, if a molar excess of the citric acid is utilized relative to the basic catalyst, a residual amount of the citric acid, which is unreacted, i.e., not neutralized, may remain in the reduced-odor polyol composition. Typically, any residual amount of the citric acid and/or the basics catalyst present in the reduced-odor polyol composition is such that the reduced-odor polyol composition maintains a pH of from 4.5 to 7.5.

The term “substantially”, as used herein in reference to embodiments in which the reduced-odor polyol composition being substantially free from antioxidants, refers to the reduced-odor polyol composition being substantially free from antioxidants discretely added to the reduced-odor polyol composition. More specifically, the reduced-odor polyol composition may comprise antioxidants other than the acid salt and optionally a residual amount of the citric acid and/or the basic catalyst in an amount typically less than 2.0, more typically less than 1.0, most typically 0.0 parts by weight based on 100 parts by weight of the reduced-odor polyol composition without departing from the definition of substantially free from antioxidants. For example, in conventional polyol compositions, antioxidants, such as vitamin E and/or butylated hydroxytoluene (BHT), are included to minimize and/or prevent oxidative degradation of the conventional polyol compositions. Such antioxidants impose additional complications with respect to quality control, as these antioxidants can have adverse effects on the physical properties of the conventional polyol compositions, including appearance and homogeneity. The reduced-odor polyol composition of the present invention has excellent physical properties and resistance to oxidative degradation even in the absence of such antioxidants discretely added to conventional polyol compositions.

As set forth above, in certain embodiments, the reduced-odor polyol composition consists essentially of the polyol and the acid salt. In these embodiments, the reduced-odor polyol composition is typically pelletized, as described in greater detail below with respect to the method of producing the reduced-odor polyol composition. In these embodiments, the reduced-odor polyol composition is typically a solid. Generally, the reduced-odor polyol composition is pelletized and shipped. In particular, the reduced-odor polyol composition is typically prilled via a prill tower for packaging and shipping purposes. Pharmaceutical companies may compound the reduced-odor polyol composition with other ingredients, or may repackage the reduced-odor polyol composition to be sold to patients along with proper dosing instructions. Typically, patients dissolve the reduced-odor polyol composition in water prior to orally consuming the reduced-odor polyol composition. Typically, when the reduced-odor polyol composition consists essentially of the polyol and the acid salt, the reduced-odor polyol composition is a white solid.

As alluded to above, the reduced-odor polyol composition of the present invention is particularly suitable for gastrointestinal lavage of a mammal. When the reduced-odor polyol composition is utilized as the gastrointestinal lavage, the reduced-odor polyol composition is typically dissolved in water for administering the reduced-odor polyol composition to the mammal such that the reduced-odor polyol composition further comprises water. In these embodiments, the reduced-odor polyol composition consists essentially of the polyol, the acid salt and the water. It is to be appreciated that the acid salt may disassociate into respective ions when the reduced-odor polyol composition further comprises water without departing from the scope of the reduced-odor polyol composition of the present invention. The reduced-odor polyol composition has a laxative effect when orally consumed, which results in gastrointestinal lavage.

In addition, the reduced-odor polyol composition has a pH of from 4.5 to 7.5, more typically from 5.25 to 7.25, most typically from 6.0 to 7.0. The reduced-odor polyol composition has a pH within these ranges whether the reduced-odor polyol composition is a solid or is dissolved in water.

When the reduced-odor polyol composition consists essentially of the polyol and the acid salt, i.e., when the reduced-odor polyol composition is a solid, the reduced-odor polyol composition has an excellent solubility in water, as evidenced by its dissolution rate. In particular, the reduced-odor polyol composition, when a solid, has a dissolution rate of less than 11, typically less than 10, more typically less than 9, and most typically less than 8 minutes and 45 seconds in water, as measured according to the method described below.

As set forth above, the present invention also provides a method of producing the reduced-odor polyol composition. The method of producing the reduced-odor polyol composition comprises the step of providing at least one alkylene oxide. Suitable alkylene oxides include, but are not limited to, ethylene oxide, propylene oxide, butylene oxide, or mixtures of these alkylene oxides. It is to be appreciated that a single type of alkylene oxide, such as ethylene oxide, may be utilized, or a blend of different types of alkylene oxides, such as ethylene oxide and propylene oxide, may be utilized. For the purposes of clarity, the at least one alkylene oxide is hereinafter referred to “the alkylene oxide”, which encompasses embodiments in which the alkylene oxide comprises the blend of different types of alkylene oxides. The alkylene oxide is utilized to polyoxyalkylate an initiator, described additionally below, to form the polyol of the reduced-odor polyol composition.

The method of producing the reduced-odor polyol composition also comprises the step of providing at least one initiator. For the purposes of clarify, the at least one initiator is hereinafter referred to “the initiator”, which encompasses embodiments in which the at least initiator comprises the blend of different types of initiators. As understood by those skilled in the art, the initiator has at least one alkylene oxide reactive hydrogen, typically at least two alkylene oxide reactive hydrogens. Suitable initiators include, but are not limited to, an alcohol, a polyhydroxyl compound, a mixed hydroxyl and amine compound, an amine, a polyamine compound, an imine, an acid, or mixtures of these initiators. Examples of alcohols that may be utilized as the initiator include, but are not limited to, glycols, such as ethylene glycol and propylene glycol, and aliphatic and aromatic alcohols, such as lauryl alcohol, nonylphenol, octylphenol and C₁₂ to C₁₈ fatty alcohols. Examples of the polyhydroxyl compounds that may be utilized as the initiator include, but are not limited to, diols, triols, and higher functional alcohols such as sucrose and sorbitol. Examples of amines that may be utilized as the initiator include, but are not limited to, aniline, dibutylamine, and C₁₂ to C₁₈ fatty amines. Examples of polyamine compounds that may be utilized as the initiator include, but are not limited to, diamines such as ethylene diamine, toluene diamine, and other polyamines. One example of an acid that may be utilized as the initiator is carboxylic acid. Additional examples of initiators suitable for the purposes of the present invention include dipropylene glycol, trimethylene glycol, 1,2-butanediol, 1,3-butanediol, 1,4-butanediol, 1,2-pentanediol, 1,4-pentanediol, 1,5-pentanediol, 1,6-hexanediol, 1,7-heptanediol, glycerol, 1,1,1-trimethylolpropane, 1,1,1-trimethylolethane, 1,2,6-hexanetriol, α-methyl glucoside, pentaerythritol, sorbitol, aniline, o-chloroaniline, p-aminoaniline, 1,5-diaminonaphthalene, methylene dianiline, the condensation products of aniline and formaldehyde, 2,3-, 2,6-, 3,4-, 2,5-, and 2,4-diaminotoluene and isomeric mixtures, methylamine, triisopropanolamine, ethylenediamine, 1,3-diaminopropane, 1,3-diaminobutane, 1,4-diaminobutane, and combinations thereof. The initiator is typically utilized in an amount contingent on the desired molecular weight of the polyol produced by the addition of the alkylene oxide, as described in greater detail below.

The method of producing the reduced-odor polyol composition further comprises the step of reacting the alkylene oxide with the initiator in the presence of a basic catalyst to produce the polyol. The alkylene oxide and the initiator are typically reacted in a vessel at a temperature of from about 80 to about 150, more typically from about 100 to about 140, degrees Celsius. The step of reacting the alkylene oxide with the initiator takes place in the presence of the basic catalyst. The basic catalyst is typically present in the vessel in an amount such that the residue of ignition value is less than 0.1% as specified by the United States Pharmacopeia-National Formulary (USP-NF) monograph for polyethylene glycol.

After the reaction between the alkylene oxide and the initiator to produce the polyol, the basic catalyst remains unreacted in a mixture with the polyol. As set forth above, in certain embodiments, the basic catalyst comprises an alkali hydroxide selected from the group of lithium hydroxide (LiOH), sodium hydroxide (NaOH), potassium hydroxide (KOH), rubidium hydroxide (RbOH), caesium hydroxide (CsOH), and combinations thereof. Because the basic catalyst is generally undesirable for oral consumption by a mammal, the method of producing the reduced-odor polyol composition also comprises the step of neutralizing the basic catalyst with citric acid, thereby forming at least one acid salt and producing the reduced-odor polyol composition. Typically, the step of neutralizing the basic catalyst with the citric acid comprises adding the citric acid in an amount such that the reduced-odor polyol composition has a pH of from 4.5 to 7.5, more typically from 5.5 to 7.25, most typically from 6.0 to 7.0, which is suitable for oral consumption by a mammal, as described in greater detail below.

In certain embodiments, the method of producing the reduced-odor polyol composition further comprises the step the step of vacuum stripping the reduced-odor polyol composition to substantially remove any residual water, unreacted alkylene oxide and/or any other volatile impurities other than the acid salt from the reduced-odor polyol composition. For example, the reduced-odor polyol composition may contain volatile impurities, which can have potentially undesirable effects on physical properties of the reduced-odor polyol composition. Examples of volatile impurities that may be present in the reduced-odor polyol composition include unreacted monomers, organic by-products, and moisture. The step of vacuum stripping the reduced-odor polyol composition typically comprises stripping the reduced-odor polyol composition with an inert gas at a temperature of from about 90 to about 130 degrees Celsius. Examples of inert gasses suitable for the step of stripping the reduced-odor polyol composition include, but are not limited to, nitrogen, argon, and helium.

As set forth above, the reduced-odor polyol composition is typically a solid. As such, in certain embodiments, the method of producing the reduced-odor polyol composition also comprises the step of pelletizing the reduced-odor polyol composition. The reduced-odor polyol composition is typically pelletized to a desired size for shipping the reduced-odor polyol composition. The reduced-odor polyol composition may be pelletized by any apparatus known in the art, such as by a pelletizer or a prill tower, or may be pelletized by other methods.

In certain embodiments, the method of producing the reduced-odor polyol composition further comprises the step of dissolving the reduced-odor polyol composition in water. The reduced-odor polyol composition may be dissolved into water without being pelletized or after the step of pelletizing the reduced-odor polyol composition. Typically, the reduced-odor polyol composition is dissolved into water before administering the reduced-odor polyol composition to the mammal, as set forth in greater detail below. As set forth above, the reduced-odor polyol composition has excellent solubility in water, as evidenced by its dissolution rate. The dissolution rate of the reduced-odor polyol composition is dependent upon several factors, such as particle size, water temperature, stirring rate (or other rate of agitation), etc. To accurately measure the dissolution rate of the reduced-odor polyol composition relative to other conventional polyol compositions, each of these variables must be uniform. To measure the dissolution rate, the reduced-odor polyol composition is molten, poured onto aluminum foil, solidified, and flaked. The same process can be done with other polyol compositions to obtain flakes of identical size and mass for comparative purposes. Three grams of the reduced-odor polyol composition, once flaked, are placed into 30 mL of water at room temperature and stirred with a magnetic stirrer at a speed setting of 7. Solubility is determined by optical inspection. Once there is no longer any visible residue of the reduced-odor polyol composition, the reduced-odor polyol composition is designated as being dissolved in the water. One gram of the reduced-odor polyol composition, when flaked, has a dissolution rate of less than 11, typically less than 10, more typically less than 9, and most typically less than 8 minutes and 45 seconds in 10 mL of room temperature water.

As set forth above, the present invention also provides a method of treating a mammal, such as a human, with the reduced-odor polyol composition. The method of treating the mammal comprises the steps of providing the reduced-odor polyol composition and administering the reduced-odor polyol composition to treat the mammal.

Typically, the step of administering the reduced-odor polyol composition comprises introducing the reduced-odor polyol composition into a gastrointestinal tract of the mammal. The step of introducing the reduced-odor polyol composition into the gastrointestinal tract may be accomplished by orally consuming the reduced-odor polyol composition. Stated differently, the mammal, i.e., the human, drinks the reduced-odor polyol composition. As such, if the reduced-odor polyol composition is a solid, it is typically dissolved in water prior to being administered to the mammal. After ingesting the reduced-odor polyol composition by the mammal, a gastrointestinal tract of the mammal is typically discharged. As such, the step of administering the reduced-odor polyol composition is typically performed prior to and is excellent in the preparation for a gastrointestinal procedures, such as a colonoscopy. The reduced-odor polyol composition has a laxative effect, which results in gastrointestinal lavage of the mammal. Dosages are typically determined based on the desired effect of the reduced-odor polyol composition.

The following examples, illustrating the reduced-odor polyol composition and the method of producing the reduced-odor polyol composition of the present invention, are intended to illustrate and not to limit the invention.

### EXAMPLES

### Example 1

A reduced-odor polyol composition is formed by disposing 357.0 grams of an diethylene glycol (initiator) and 3.6 grams of 96.0% sodium hydroxide (basic catalyst), in a 5 gallon autoclave which is pressure purged with nitrogen. The autoclave is evacuated to remove water while being agitated and is heated to a temperature of 115° C. When the temperature of the autoclave reaches 115° C., the vacuum is released and the autoclave is pressured with nitrogen. 11,622 grams of ethylene oxide (EO) are introduced into the autoclave at a rate which keeps a pressure within the autoclave below the net safety limits of the autoclave. The temperature of the autoclave is raised to 150° C. Once the 11,622 grams of ethylene oxide (EO) have been disposed into the autoclave, the ethylene oxide and the diethylene glycol are reacted in the presence of the sodium hydroxide at a constant pressure to produce a polyol. The polyol is discharged from the autoclave into a five gallon pail. A 5% aqueous solution of the polyol has a pH of 10.2, which is attributable to the unreacted basic catalyst, i.e., the sodium hydroxide present along with the polyol.

To neutralize the basic catalyst, 1,500 grams of the polyol are disposed in a 2 liter 4-necked flask equipped with a heating mantle and agitator. The flask is heated to about 85-95° C. with agitation. 1.58 grams of 50% citric acid are added to the flask to neutralize the basic catalyst, thereby producing the reduced-odor polyol composition. Water and any other volatile impurities are stripped by evacuating the flask under agitation for 30 minutes.

To calculate a dissolution rate of the reduced-odor polyol composition, the reduced-odor polyol composition is molten, poured onto aluminum foil, and cracked into flakes. The dissolution rate of the reduced-odor polyol composition is calculated by dissolving 3 grams of the reduced-odor polyol composition, when flaked, in 30 mL of water. The reduced-odor polyol composition is dissolved via a magnetic stirrer having a speed setting of 7 in a beaker. Solubility is determined by optical inspection. Once there is no longer any visible residue of the reduced-odor polyol composition, the reduced-odor polyol composition is designated as being dissolved in the water. The dissolution rate of the reduced-odor polyol composition is set forth in Table 1 below.

TABLE 1

| Neutralization | Dissolution Time: |
|---|---|
| Acid: | (g/10 mL water) |
| Citric Acid | 520 seconds |

Odor of the reduced-odor polyol composition is determined by a panel of three persons. To determine the odor of the reduced-odor polyol composition, the reduced-odor polyol composition is placed in a brown jar and stored for three months. After three months of storage, each of the panelists independently and blindly smells the reduced-odor polyol composition and designates the odor on a scale of 1 through 5, in which 4-5 is a strong odor, 2-3 is a medium odor, less than 2 is mild, with a rating of 1 being the most minimal and/or negligible odor. For a comparative basis, a conventional polyol composition which is neutralized by a mixture of acetic and phosphoric acids is the control odor rating and designated as a 5 on the scale. The odor rating by each panelist, as well as the average rating for the three panelists, for the reduced-odor polyol composition is illustrated in Table 2 below:

TABLE 2

| | Panelist 1 | Panelist 2 | Panelist 3 | Average |
|---|---|---|---|---|
| Polyol Composition | Rating | Rating | Rating | Rating: |
| Reduced-Odor Polyol | 1 | 1 | 1 | 1.00 |
| Composition | | | | |

### Comparative Examples

Polyol compositions 1-5 are prepared in the same manner as Example 1 with the exception that different acids are utilized to neutralize the basic catalyst.

### Polyol Composition 1

1,503 grams of the polyol formed in Example 1 are disposed in a 2 liter 4-necked flask equipped with a heating mantle and agitator. The flask is heated to about 90° C. with agitation. Carbon dioxide gas is injected into the flask from a carbon dioxide cylinder to neutralize the basic catalyst until the pH of a 5% aqueous solution of the polyol measured about 6.22.

### Polyol Composition 2

1,500 grams of the polyol formed in Example 1 are disposed in a 2 liter 4-necked flask equipped with a heating mantle and agitator. The flask is heated to about 85-95° C. with agitation. 1.19 grams of 85% lactic acid are disposed in the flask to neutralize the basic catalyst. Water and any other volatile impurities are stripped by evacuating the flask under agitation for 30 minutes.

### Polyol Composition 3

1,500 grams of the polyol formed in Example 1 are disposed in a 2 liter 4-necked flask equipped with a heating mantle and agitator. The flask is heated to about 85-95° C. with agitation. A combination of 50% citric acid and 85% phosphoric acid are disposed in the flask to neutralize the basic catalyst in an amount such that a 5% aqueous solution of the polyol composition has a pH of 6.44. Water and any other volatile impurities are stripped by evacuating the flask under agitation for 30 minutes.

### Polyol Composition 4

1,500 grams of the polyol formed in Example 1 are disposed in a 2 liter 4-necked flask equipped with a heating mantle and agitator. The flask is heated to about 85-95° C. with agitation. A combination of 85% lactic acid and 85% phosphoric acid are disposed in the flask to neutralize the basic catalyst in an amount such that a 5% aqueous solution of the polyol composition has a pH of 4.95. Water and any other volatile impurities are stripped by evacuating the flask under agitation for 30 minutes.

### Polyol Composition 5

1,500 grams of the polyol formed in Example 1 are disposed in a 2 liter 4-necked flask equipped with a heating mantle and agitator. The flask is heated to about 85-95 ° C. with agitation. 0.87 grams of 85% phosphoric acid are disposed in the flask to neutralize the basic catalyst. Water and any other volatile impurities are stripped by evacuating the flask under agitation for 30 minutes.

### Conventional Polyol Composition

Conventional Polyol Composition is commercially available and formed via the same method as described in Example 1 with the exception that a combination of acetic acid and phosphoric acid are utilized to neutralize the basic catalyst.

The dissolution rates of polyol compositions 1-5 are set forth in Table 3 below, and are calculated by the same method as the reduced-odor polyol composition. In particular, polyol compositions 1-5 are flaked in a similar manner as the reduced-odor polyol composition, and the dissolution rates are calculated with all variables being constant (such as mass of each respective polyol composition, water temperature, water volume, and stirring rate). In instances in which a combination of acids is utilized to neutralize the basic catalyst, the phosphoric acid is a limiting acid which neutralizes 45% of the basic catalyst on an equivalent basis. In addition, each of the dissolution rates for polyol compositions 1-6 are calculated simultaneously with the dissolution rate of the conventional polyol composition. Stated differently, the dissolution rate of the conventional polyol composition is measured each time the dissolution rate for each of the polyol compositions 1-5 is measured.

TABLE 3

| | | Dissolution Time of |
|---|---|---|
| | | Conventional Polyol |
| | Dissolution Time: | Composition |
| Polyol Composition | (g/10 mL water) | (g/10 mL water) |
| Polyol Composition 1 | 597 seconds | 525 second |
| Polyol Composition 2 | 523 seconds | 576 seconds |
| Polyol Composition 3 | 561 seconds | 590 seconds |
| Polyol Composition 4 | 540 seconds | 576 seconds |
| Polyol Composition 5 | 545 seconds | 545 seconds |

As evidenced by Table 3, the dissolution time of polyol compositions 1-5 is between 523 and 597 seconds per gram of each respective polyol composition in 10 mL of water. In addition, the dissolution time of the conventional polyol composition ranged from 525 to 590 seconds per gram/10 mL of water. The dissolution time of the reduced-odor polyol composition of the subject invention was 520 seconds, which is faster than any of polyol compositions 1-5 and the conventional polyol composition, and significantly faster than a majority of these polyol compositions.

Odor of these polyol compositions is determined by the same panel and by the same method as the reduced-odor polyol composition. The odor rating by each panelist, as well as the average rating for the three panelists, for polyol compositions 1-5 and the conventional polyol composition is illustrated in Table 4 below:

TABLE 4

| | Panelist 1 | Panelist 2 | Panelist 3 | Average |
|---|---|---|---|---|
| Polyol Composition | Rating | Rating | Rating | Rating: |
| Polyol Composition 1 | 4 | 3 | 4 | 3.67 |
| Polyol Composition 2 | 3 | 3 | 3 | 3.00 |
| Polyol Composition 3 | 2 | 2 | 2 | 2.00 |
| Polyol Composition 4 | 2 | 2 | 2 | 2.00 |
| Polyol Composition 5 | 3 | 3 | 3 | 3.00 |
| Conventional Polyol | 5 | 5 | 5 | 5.00 |
| Composition | | | | |

Because each of polyol compositions 1-5 are susceptible to oxidative degradation, as evidenced by their undesirable odors after three months of storage, the effect of an antioxidant on each of these polyol compositions to reduce oxidative degradation is tested. As such, polyol compositions 6-9 below each include 100 parts per million (ppm) of vitamin E as an antioxidant. Polyol composition 6 is the same as polyol composition 2 but includes 100 ppm of vitamin E. Polyol composition 7 is the same as polyol composition 3 but includes 100 ppm of vitamin E. Polyol composition 8 is the same as polyol composition 4 but includes 100 ppm of vitamin E. Polyol composition 9 is the same as polyol composition 5 but includes 100 ppm of vitamin E. Odor of polyol compositions 6-9 is determined by the same panel and by the same method as above. The odor rating by each panelist, as well as the average rating for the three panelists, for polyol compositions 6-9 is illustrated in Table 5 below:

TABLE 5

| | Panelist 1 | Panelist 2 | Panelist 3 | Average |
|---|---|---|---|---|
| Polyol Composition | Rating | Rating | Rating | Rating: |
| Polyol Composition 6 | 3 | 3 | 2 | 2.67 |
| Polyol Composition 7 | 2 | 3 | 3 | 2.67 |
| Polyol Composition 8 | 2 | 2 | 2 | 2.00 |
| Polyol Composition 9 | 3 | 3 | 3 | 3.00 |

As evidenced by Tables 4 and 5, in certain instances, the addition of vitamin E decreases the odor associated with the polyol compositions. For Example, as set forth above, polyol composition 6 is the same as polyol composition 2 but includes vitamin E. Polyol composition 2 had an average odor rating of 3.00, whereas polyol composition 6 has an average odor rating of 2.67, which is a decrease in the odor caused by oxidative degradation. However, it is notable that even with the decrease of the odor associated with these polyol compositions, none of polyol compositions 1-9 have an odor which is as minimal as the odor of the reduced-odor polyol composition, which had a unanimous odor rating of 1.00. The unanimous odor rating of 1.00 is achieved in the reduced-odor polyol composition without vitamin E, which decreases the processing steps and cost of producing the reduced-odor polyol composition as compared to polyol compositions 6-9.

The present invention has been described herein in an illustrative manner, and it is to be understood that the terminology which has been used is intended to be in the nature of words of description rather than of limitation. Obviously, many modifications and variations of the present invention are possible in light of the above teachings. The invention may be practiced otherwise than as specifically described above.

1-11. (canceled)

12. A method of producing a reduced-odor polyol composition for gastrointestinal lavage of a mammal, said method comprising the steps of:
providing at least one alkylene oxide;
providing at least one initiator;
reacting the at least one alkylene oxide with the at least one initiator in the presence of a basic catalyst to produce a polyol; and
neutralizing the basic catalyst with citric acid, thereby forming at least one acid salt and producing the reduced-odor polyol composition;
wherein the reduced-odor polyol composition comprises the polyol and the at least one acid salt when introduced into a gastrointestinal tract of the mammal.

13. A method as set forth in claim 12 wherein the reduced-odor polyol composition is a solid.

14. A method as set forth in claim 12 wherein one gram of the reduced-odor polyol composition has a dissolution rate of less than 9 minutes in 10 mL of room temperature water.

15. A method as set forth in claim 12 wherein the reduced-odor polyol composition consists essentially of the polyol and the acid salt.

16. A method as set forth in claim 12 wherein the reduced-odor polyol composition is substantially free from antioxidants other the acid salt and optionally a residual amount of the citric acid and/or the basic catalyst.

17. A method as set forth in claim 12 further comprising the step of vacuum stripping the reduced-odor polyol composition to substantially remove any water and/or volatile impurities other than the acid salt from the reduced-odor polyol composition.

18. A method as set forth in claim 12 further comprising the step of pelletizing the reduced-odor polyol composition.

19. A method as set forth in claim 12 further comprising the step of dissolving the reduced-odor polyol composition in water.

20. A method as set forth in claim 19 wherein the reduced-odor polyol composition consists essentially of the polyol, the acid salt and the water.

21. A method as set forth in claim 12 wherein the polyol of the reduced-odor polyol composition is further defined as a polyalkylene glycol.

22. A method as set forth in claim 21 wherein the polyalkylene glycol is further defined as a polyethylene glycol having a nominal average molecular weight of from 3,000 to 3,700.

23. A method as set forth in claim 12 wherein the at least one initiator is selected from the group of an alcohol, a polyhydroxyl compound, a mixed hydroxyl and amine compound, an amine, a polyamine compound, an imine, an acid, and combinations thereof.

24. A method as set forth in claim 12 wherein the at least one alkylene oxide is selected from the group of ethylene oxide, propylene oxide, butylene oxide, and combinations thereof.

25. A method as set forth in claim 12 wherein the basic catalyst is further defined as an alkali hydroxide.

26. A method as set forth in claim 12 wherein the step of neutralizing the catalyst with the citric acid comprises adding the citric acid to the catalyst such that the reduced-odor polyol composition has a pH of from 4.5 to 7.5.

27. A method comprising the steps of:
providing at least one alkylene oxide;
providing at least one initiator;
reacting the at least one alkylene oxide with the at least one initiator in the presence of a basic catalyst to produce a polyol; and
neutralizing the basic catalyst with citric acid to form at least one acid salt, thereby producing a reduced-odor polyol composition which comprises the polyol and the at least one acid salt; and
administering the reduced-odor polyol composition to a mammal.

28. A method as set forth in claim 27 wherein one gram of the reduced-odor polyol composition in solid form has a dissolution rate of less than 9 minutes in 10 mL of room temperature water.

29. A method as set forth in claim 27 wherein the reduced-odor polyol composition is substantially free from antioxidants other than the acid salt and optionally a residual amount of the citric acid and/or the basic catalyst.

30. A method as set forth in claim 27 wherein the reduced-odor polyol composition is dissolved in water.

31. A method as set forth in claim 30 wherein the reduced-odor polyol composition consists essentially of the polyol, the acid salt, and the water.

32. A method as set forth in claim 27 wherein the step of administering the reduced-odor polyol composition to treat the mammal comprises introducing the reduced-odor polyol composition into a gastrointestinal tract of the mammal.

33. A method as set forth in claim 27 wherein the reduced-odor polyol composition has a pH of from 4.5 to 7.5.

34. A method as set forth in claim 27 wherein the polyol is further defined as a polyalkylene glycol.

35. A method as set forth in claim 34 wherein the polyalkylene glycol is further defined as a polyethylene glycol having a nominal average molecular weight of from 3,000 to 3,700.

36. A reduced-odor polyol composition formed in accordance with the method of claim 12.
